(19) United States
(12) Patent Application Publication
(10) Pub. No.: US 2019/0096000 A1
(43) Pub. Date: Mar. 28, 2019

(54) System and Method for Sharing User Information with an Insurer Utilizing Wireless Earpieces

(71) Applicant: BRAGI GmbH, Munchen (DE)

(72) Inventor: Peter Vincent Boesen, München (DE)

(73) Assignee: BRAGI GmbH, München (DE)

(21) Appl. No.: 16/131,813

(22) Filed: Sep. 14, 2018

Related U.S. Application Data
(60) Provisional application No. 62/562,985, filed on Sep. 25, 2017.

(51) Int. Cl. G06Q 40/08; H04R 1/10; A61B 5/0205; A61B 5/00; A61B 5/1171

(52) U.S. Cl. CPC G06Q 40/08; H04R 1/1016; H04R 1/1091; A61B 5/0205; A61B 5/02438; A61B 5/0022; A61B 5/1171; A61B 5/4803; H04R 2420/07; A61B 5/6817

(57) ABSTRACT

A system, method and wireless earpiece for sharing user data with an insurance provider to provide an insurance product of adjust the cost of insurance. The wireless earpiece may establish the identity of a user and track activity data of the user using at least a heart rate sensor and an inertial sensor of the wireless earpiece. The activity data and user identity may be wireless communicated to a server platform of the insurance provider. The server platform may apply the user identity and activity data to an actuarial model used by the insurance provider. The user may be provided a product based on the actuarial model utilizing the activity data and user identity.

### PRIORITY STATEMENT

This application claims priority to 62/562,985, filed Sep. 25, 2017, which is hereby incorporated by reference in its entirety.

### FIELD OF INVENTION

This present invention relates to wireless earpieces. More specifically, but not exclusively, the present invention relates to sharing user information with an insurer utilizing biometric readings and other information from the wireless earpieces.

### BACKGROUND

One recognized use of wearable devices such as wireless earpieces is to provide biometric monitoring of a user in one form of another. However, the collection of such data by a set of wireless earpieces or other wearable devices may have limited utility. Such data may, in some cases, not be stored. Or if such data is stored, it may be stored only in a data silo. That is to say the data store for such data may be isolated and segregated from other data. In addition, such data may not always be accurate and complete.

Such data, however, may be rich in context and highly informative to insurance providers for determining the best life insurance or health insurance product for the user. What is a needed is a better way to use biometric data from wireless earpieces or other wearable devices in an insurance context.

### SUMMARY

Therefore, it is a primary object, feature, or advantage of the illustrative embodiments to improve over the state of the art.

It is a further object, feature, or advantage of the illustrative embodiments to provide opportunities for insured individuals to reduce insurance premiums for health insurance, life insurance, and other types of insurance by providing data indicative that such individuals are at a lower risk of death, incurring significant health care costs, or other insured risk.

It is a still further object, feature, or advantage of the illustrative embodiments to provide insurers with additional activity data which may be used to improve and refine an actuarial model.

It is a still further object, feature, or advantage of the illustrative embodiments to allow a user to reduce insurance premiums for healthy behavior using activity data sensed by a wireless earpiece.

Another object, feature, or advantage is to allow a user to reduce insurance premiums for healthy behavior by incorporating activity data sensed by other wearables in addition to the wireless earpiece.

Yet another object, feature, or advantage is to provide feedback to a user if the user either satisfies or fails to satisfy a condition for reducing an insurance premium.

One aspect of the present invention includes a method for sharing user information with an insurer utilizing a server platform associated with an insurance provider and a wireless earpiece. The method may comprise of establishing the identity of a user using the wireless earpiece. The method may further include tracking the activity of the user using at least an inertial sensor of the user and a heart rate sensor of the user. The method may also include wirelessly communicating the identity of the user and the activity of the user to a server platform. Additionally, the method may include applying the activity of the user to an actuarial model at the server platform, wherein an insurance product is provided to the user based on the actuarial model. The method may further comprise providing feedback to the user through the wireless earpiece indicative of progress towards an activity goal based on the activity of the user wherein the activity goal is associated with an insurance adjustment. The method may also include communicating from a transceiver of the wireless earpiece the activity of the user to a mobile application on a computing device of the user. The data may be communicating from the mobile application on the computing device of the user over a wireless communications network to the server platform. The method may also include communicating activity goals from the server platform through a wireless network to the computing device of the user. The method may include associating activity data communicated from the wireless earpiece to the computing device with the activity goals stored on the mobile application.

One or more of the following features may be included. A processor of the wireless earpiece may be programmed to process the activity data in accordance with one or more parameters determined by the insurance provider related to the condition for the premium adjustment. An air microphone may be connected to the processor and face away from the user and configured to receive ambient sounds and verbal commands from the user. The ambient sounds may be compared by the processor for determining a user action related to the condition. The verbal commands may include responses to queries of an insurance evaluation. The wireless earpiece may track activity of a user while the user is exercising. The wireless earpiece may establish the identity of the user using biometric data received by at least one sensor of the wireless earpiece such as a voice characteristic. The biometric data may include data received from a heart sensor of the wireless earpiece. The biometric data may also include a topographical map of the portion of an ear of the user. The insurance product provided to the user may include a life insurance product or a health insurance product. A processor of the wireless earpiece may analyze the activity data. The processor may determine whether the activity data of the user satisfies an activity goal communicate from the server platform.

In another aspect, a method for sharing user information with an insurance provider includes receiving, at a sensor associated with a wireless earpiece, activity data associated with a user, and communicating, via a wireless transceiver associated with the wireless earpiece, the activity data to the insurance provider if the activity data is related to a condition associated with the premium adjustment.

One or more of the following features may be included. Secondary activity data may be received from at least one additional wearable worn by the user. A processor associated with the wireless earpiece may process the activity data. The processor associated with the wireless earpiece may also process the secondary activity data. The processing may be performed in accordance with one or more activity goals set by the insurance provider. The activity goal associated with the adjust of an insurance product or an insurance product may include exercising for a specific time period. One or more activity data associated with the activity goal may be selected from a group consisting of blood pressure, heartrate, blood oxygen level, blood sugar level, and a respiration rate. The insurance provider may be notified if the user fails to satisfy the activity goal A speaker associated with the wireless earpiece may communicate a suggestion for satisfying the activity goal. A warning may be provided to the user if the activity data is outside of a prescribed range for satisfying the condition associated with the activity goal. A suggestion may be provided to the user if the activity data is outside of a prescribed range for satisfying the activity goal.

In another embodiment, a system includes a wireless earpiece and an server platform. The wireless earpiece includes an earpiece housing, a processor disposed within the earpiece housing, a sensor connected to the processor and configured to sense activity data of a user, and a wireless transceiver connected to the processor. The server platform includes a user profile associated with the user having identification information, policy information, and premium adjustment conditions. The wireless earpiece transmits a signal encoding the activity data to the server platform in response to a completion of an action by the user indicative of a condition for receiving a premium adjustment.

One or more of the following features may be included. The signal transmitted by the wireless earpiece may be transmitted at a specific frequency provided by the server platform to establish a connection to the server platform. The server platform may transmit a second signal to the wireless earpiece if the signal transmitted by the wireless earpiece was not received by the server platform. The server platform may transmit a signal encoding updates to parameters required to satisfy the condition for receiving the premium adjustment.

One or more of these and/or other objects, features, or advantages of the illustrative embodiments will become apparent from the specification and claims that follow. No single embodiment need provide each and every object, feature, or advantage. Different embodiments may have different objects, features, or advantages. Therefore, the illustrative embodiments not to be limited to or by an object, feature, or advantage stated herein.

### BRIEF DESCRIPTION OF THE DRAWINGS

FIG. 1 illustrates a pictorial representation of a pair of wireless earpieces and a server platform.

FIG. 2 illustrates another aspect of a pair of wireless earpieces and a server platform.

FIG. 3 is a block diagram of a wireless earpiece.

FIG. 4 illustrates a pair of wireless earpieces.

FIG. 5 illustrates a right earpiece and its relationship with a user's right ear in accordance with an illustrative embodiment.

FIG. 6. illustrates a user information portfolio including profile information, activity data, and policy information.

FIG. 7 illustrates an actuarial model used in the offering of an insurance product.

FIG. 8 illustrates a method for sharing user information with an insurance provider.

FIG. 9 illustrates a flowchart of another aspect of sharing user information with an insurance provider.

### DETAILED DESCRIPTION

The invention is not to be limited to the particular embodiments described herein. In particular, the invention contemplates numerous variations in populating electronic medical records (EMRs) using wearable devices. The foregoing description has been presented for purposes of illustration and description. It is not intended to be an exhaustive list or limit any of the invention to the precise forms disclosed. It is contemplated that other alternatives or exemplary aspects are considered included in the invention. The description is merely examples of embodiments, processes or methods of the invention. It is understood that any other modifications, substitutions, and/or additions can be made, which are within the intended spirit and scope of the invention.

The illustrative embodiments provide a system, method, and wireless earpieces for sharing activity data and user information with an insurance provider. The wireless earpieces may share activity data with the insurance provider utilizing information and biometrics, inertial data, biological data, physiological data, or environmental data measured by the wireless earpieces. The insurance product or cost of an insurance product for a user may be adjusted initially or adjusted based on the activity data shared with the insurer such as biometrics, environments, and so forth. For example, the insurance may be adjusted based on health/exercise, driving history, environmental conditions, and so forth. The activity data may be input into an actuarial model on a mobile app or a server platform of the insurance provider to determine what insurance premium or rate the user qualifies for or would best fit the user's lifestyle.

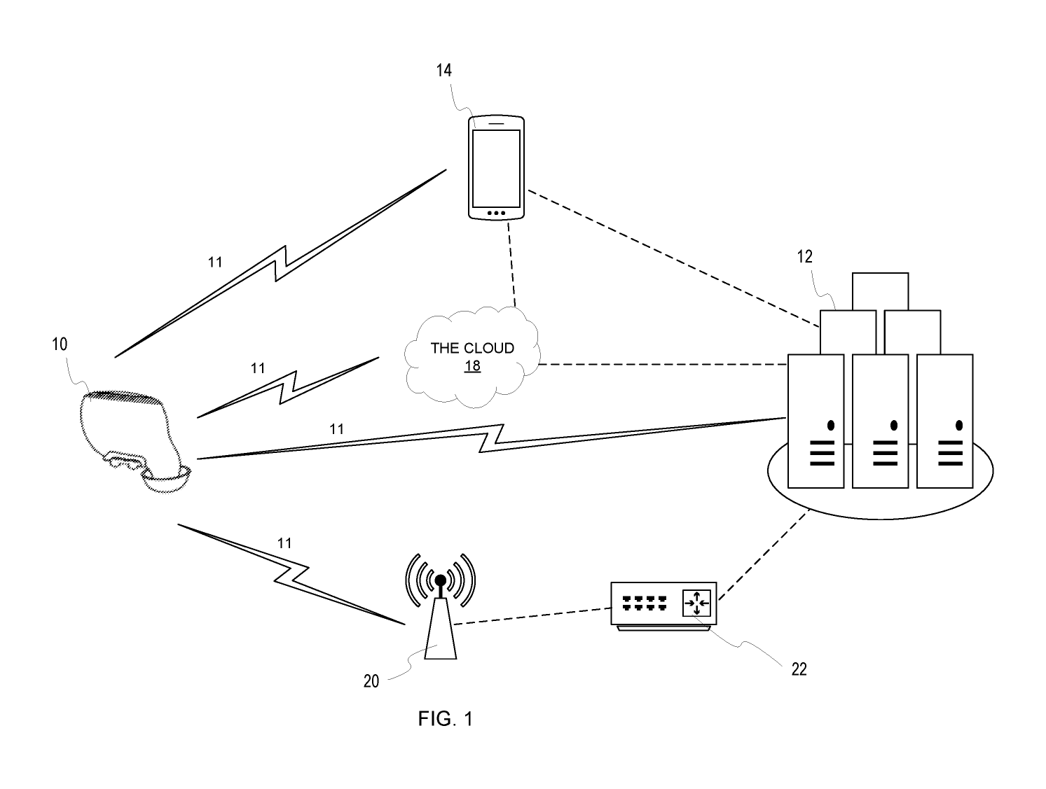

FIG. 1 illustrates a pictorial representation of one aspect of the communication system 100 of a pair of wireless earpieces 10 and a server platform 12. The wireless earpiece 10 is representative of one of a pair of wireless earpieces that may be worn by a user. For example, a first wireless earpiece may be a left wireless earpiece, and a second wireless earpiece may be a right wireless earpiece. The wireless earpiece 10 may communicate with the server platform 12 directly through the wireless transceiver. The wire earpiece 10 may also communicate with a mobile application on a computing device 14 such as a mobile phone. The mobile phone may contain a mobile application 16. The mobile application 16 may collect, organize and communicate activity data 26 such as user identity and activity data through the cloud 18 to the server platform 12.

The server platform 12 may contain one or more user information portfolios with which the user, activity data, and environmental data transmitted by the wireless earpiece 10 may be associated. The wireless signals shown may represent direct or indirect communications through one or more networks (e.g., cloud, Wi-Fi, cellular, mesh, local area network, etc.), devices, nodes, servers, or so forth.

The server platform 12 is a computer or computer program that manages access to a centralized resource or service in a network. The server platform 12 may also include programs and algorithms for processing the user, health, and environmental related data transmitted by the wireless earpiece 10. The server platform 12 may be connected to a wireless communications network (the cloud) 18.

In another aspect, the wireless earpiece 10 may transmit the data to a transceiver 20 associated with an insurance provider, which may subsequently transmit the data to a router 22 associated with the server platform 12. In one embodiment, the server platform 12 may be integrated with one or more of the routers 22, the transceiver 20, one or more logic engines, memories, databases, converters, and so forth. The signal encoding the data transmitted by the wireless earpiece 10 may be transmitted as a packet or data construct and/or at a specific frequency, phase, or waveform for connection or security purposes.

Upon receiving transmissions from the wireless earpiece 10, the server platform 12 may instruct a transceiver 20 to transmit a signal encoding a notification that the server platform 12 successfully received the data. In another embodiment, the server platform 12 may instruct the transceiver 20 to transmit a signal encoding a notification that the server platform 12 has not received the data or any other required data from the wireless earpiece 10 and to retransmit the information. The notification may also encode information relating to data routes, connections, links, security, frequencies, bandwidth requirements, network outage times, or other information pertinent to transmitting information.

In addition, information may be transmitted by the server platform 12 in response to a query from the user via the wireless earpiece 10 or another device capable of communicating with the server platform 12. For example, if the user wants to know whether the insurance provider has updated any conditions related to obtaining insurance, adjustments, pre-qualifications, incentives, rejections, or so forth, the server platform 12 may transmit a signal encoding an affirmative or a negative response or the server platform 12 may transmit a signal with updates for the insurance information. The signal may be transmitted continuously or discretely as necessary.

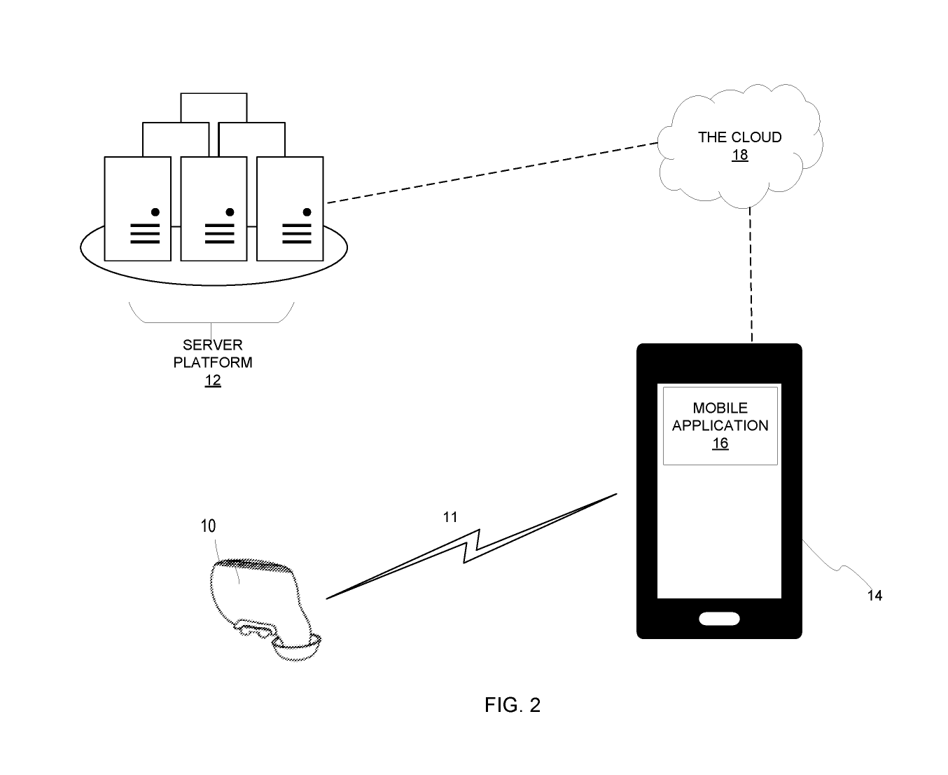

FIG. 2 shows another aspect of the communication system 100 of a pair of wireless earpieces 10 and a server platform 12. The wireless earpiece 10 may collected activity data 26 such as biometric data and environmental data and wirelessly transmit the data to the computing device 14. The computing device 14 may be a smartphone, tablet, personal computer, etc. The computing device 14 may contain a mobile application 16. The mobile application 16 may be provided by the insurance provider to collect and communicate activity data 26. The mobile application may also store policy information, billing information, user identification information, request roadside assistance, submit photos of documents, communicate with an insurance agent. etc. The mobile application may also track a user's activity using data received from the wireless earpiece and any other devices that may transmit activity data, caloric and nutritional intake, monitor and record biometric data received from the wireless earpiece. The mobile application may also input data into an actuarial model 24 used by the insurance provider.

The mobile application 16 on the computing device 14 may receive policy information and activity goals from the server platform 12 associated with an insurance provider. The mobile application may communicate user identity, activity data, and activity goal progress to the server platform 12.

The computing device 14 may utilize short-range or long-range wireless communications to communicate with the wireless earpieces 10 through the wireless signal 11 or devices of the communications system 100 through the wireless signal 11. For example, the mobile device 14 may include a Bluetooth, and cellular transceiver within the embedded logical components. For example, the wireless signal 11 may be a Bluetooth, Wi-Fi, NFMI, Zigbee, Ant+, or other short range wireless communication.

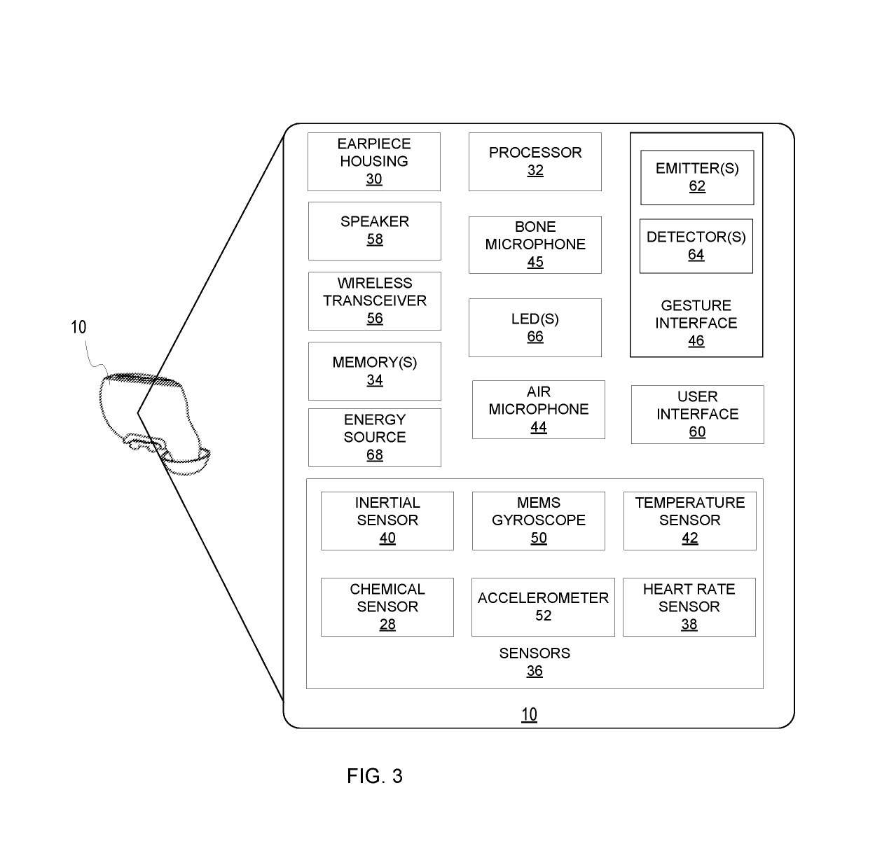

FIG. 3 illustrates one aspect of the wireless earpiece 10. The wireless earpiece may be used to track a user's activity, biometric data, environmental data or any other data an insurance provider may need to calculate or adjust a user's premium. The description of the components, structure, functions, and other elements of the wireless earpieces 10 may refer to a left wireless earpiece, a right wireless earpiece, or both wireless earpieces 10 as a set or pair. All or a portion of the components shown for the wireless earpieces 10 may be included in each of the wireless earpieces. For example, some components may be included in the left wireless earpiece, but not the right wireless earpiece and vice versa. In another example, the wireless earpieces 10 may not include all the components described herein for increased space for batteries or so forth.

The wireless earpiece 10 may include an earpiece housing. The earpiece housing 30 is a structure shaped to fit substantially within the ear of the user. The earpiece housing 30 may at least partially enclose one or more of the components of the wireless earpiece 10 and may be composed of one or more plastics, one or more metals, one or more polymers, one or more non-metals, or any material or combination of materials having substantial deformation resistance to facilitate energy transfer if a sudden force is applied to the wireless earpiece 10. One or more sleeves may be provided with the wireless earpiece 10 that may be fitted over a portion of the earpiece housing 30. The sleeves may come in various shapes and sizes and may be used to improve the fit of the wireless earpiece 10 within an ear of a user, improve audio transparency, improve the longevity of the wireless earpiece 10, protect the user from skin allergies, and so forth. The earpiece housing 30 or the sleeves may also be custom fitted based on the size and shape of the ears of the user.

A processor 32 is disposed within the earpiece housing 30 and operatively connected to each of the electrical components of the wireless earpiece 10. The processor 32 may be a digital integrated circuit, an analog integrated circuit, a mixed integrated circuit, an application-specific integrated circuit, an intelligent control unit, a central processing unit, or another type of logic engine or component capable of processing data, instructions, applications, and/or information, and more than one of the aforementioned types may be integrated together. The processor 32 may include logic circuitry, which may consist of combinational and/or sequential digital logic, for controlling one or more functions of the wireless earpiece 10. The processor 32 may also include a register with data and/or instructions stored thereon for further controlling the wireless earpiece 10. In addition, data and/or information stored in one or more cache memories within the processor 32 may be used by the logic circuitry to enhance the functionality of the wireless earpiece 10.

Furthermore, the processor 32 may be programmed to execute one or more kernels, applications, programs, and/or instructions to control the wireless earpiece 10 or process data or information received from one or more of the components of the wireless earpiece 10. The kernels, applications, programs, and/or instructions used by the processor 32 may be stored in one or more memories 34, the register, caches, or other memory storage devices whether integrated or separate from the processor 32. Data received from the wireless earpiece components may also be stored in one or more of the memories 34, the register, or one or more of the cache memories. For example, the processor 32 may execute an application stored in its register to process heart rate data sensed by one or more of the sensors 36. The processor 32 may also execute additional programs and/or use additional data, such as coronary data related to the user, to process the heart rate data. As another example, the processor 32 may execute a program for transmitting a signal encoding activity data sensed by one or more of the components of the wireless earpiece 10 to a server associated with an insurance provider. The measured data and signal may encode biometric data, such as blood sugar level, average heart rate, blood pressure, blood oxygen level, and so forth. The sensors may also capture activity/exercise data, such as the time the user spent walking or jogging, time spent standing, sitting, or laying, nutrition data, such as what the user eats and drinks, or other health-related data that may be of use to an insurance provider. Furthermore, the signal may encode other information, such as the date and time of transmission, the identity of the user, a connection frequency, identification, security key, or waveform for establishing a connection to the server platform 12, or other information which may be of relevance to the insurance company. The signals may be transmitted by the wireless earpiece 10 (or an associated wireless device) under a wireless signal, such as a Bluetooth, WiFi, cellular, and other wireless standards, protocols, and signals.

One or more sensors 36 are operatively connected to the processor 32 and may be used for sensing health related data and environment data for use by an insurance provider, auditor, or group. In one embodiment, the sensors 36 that may be operatively connected to the processor 32 include a heart rate sensor 38, an inertial sensor 40, a temperature sensor 42, and a chemical sensor 28. This information may provide information regarding the general well-being of the user. For example, the heart rate sensor 38 and inertial sensor 40 can track a user and collect biometric data while a user is jogging. In one example, the heart rate may be recorded continuously (such as a graph of heart rate versus time) or discretely (such as taking heart rate recording every thirty seconds) depending on the program settings, which may be set by the user via a voice command via an air microphone 44 or a gesture via a gesture interface 46. The inertial sensor 40 may include a MEMS gyroscope or an electronic accelerometer to track the movement of a user. The wireless earpiece 10 may contain additional optical sensors 48 to measure pulse oximetry and blood pressure of the user during a jogging session. This information may provide information regarding the general well-being of the user.

An inertial sensor 40, such as the MEMS gyroscope 50 or the electronic accelerometer 52, may be used to determine the orientation of the user and a type of activity the user is participating in. For example, if the processor 32 determines that the movements sensed by the inertial sensor 40 are likely to correspond with walking, the processor 32 may associate activity data sensed by additional sensors such as the heart rate sensor 38 with walking and store the combined data set in one of the memories 34 or within another component of the processor 32 for future use. The wireless earpiece 10 may include one or more motion sensors 54 which may include one or more radar sensors for measuring biometrics and the user's environment. For example, the radar sensor may be utilized to measure blood velocity, pulse rate, and so forth. The radar sensor may also measure a jogging rate, proximity to other users and objects, and so forth.

The temperature sensor 42 may measure the temperature of the user and/or the environment. The temperature sensor 42 may determine whether the user is experiencing unusual body or environmental temperatures that may affect applicable insurance premiums, deductibles, adjustments, qualifications, or so forth. As is well known, extreme body temperatures may be associated with infection, illness, disease, or other severe conditions.

The chemical sensor 28 may measure chemicals or materials of the user and/or environment. The chemical sensor 28 may utilize air samples, excretions (e.g., sweat, blood, ear wax, etc.), tissue, or other samples to determine the applicable chemical analysis.

The wireless earpiece 10 may include any number of other sensors including radiation sensors, wind sensors, barometer, altimeter, humidity, blood sensors, cameras, thermal imagers, X-rays, or so forth.

As noted any of the readings of the sensors 36 may be made continuously, periodically, based on an event or conditions, based on an application request, or so forth. The measured data may be saved, analyzed, or communicated in real-time or discretely. For example, based on analysis of sensor data processed by the processor 32, the raw or compiled information may be sent to applicable servers, platforms, equipment, computing devices, or users.

A wireless transceiver 56 is operatively connected to the processor 32 and may be used by the processor 32 to transmit signals encoding activity data measured by the sensors 36 to an insurance provider. The wireless transceiver 56 may be a Bluetooth transceiver, a WiMax transceiver, a cellular transceiver, a satellite transceiver, a Wi-Fi transceiver, or another type or class of wireless transceiver that can simultaneously receive signals from electronic devices at substantial distances and meet one or more IEEE standards. The wireless transceiver 56 may also include a near-field magnetic induction (NFMI) transceiver for sending short range signals to another electronic device (such as another wireless earpiece). The wireless transceiver 56 may be configured to receive signals from devices, such as smartphones, communications towers, satellites, desktops, laptops, watches, server platforms, insurance routers, insurance switches, or other types of electronic devices and communicate the signals to the processor 32. The processor 32 may use the data encoded in the signals during execution of one or more programs or applications and/or store the data in a memory 34. In one embodiment, the wireless transceiver 56 is a multi-mode transceiver or hybrid transceiver. For example, the wireless earpieces 10 may be configured for at least simultaneous NFMI communications to another wireless earpiece and Bluetooth communications to an associated smart phone.

One or more memories 34 may be operatively connected to the processor 32 for storing the activity data measured by the sensors 36. Each memory 34 may be either static or dynamic for allowing data storage when the wireless earpiece 10 is not actively powered. In some embodiments, additional volatile memories such as random-access memories may be incorporated into the memories 34 to allow for improved functionality. Each memory 34 may be configured and/or programmed to store kernels, applications, programs, algorithms, instructions and/or data for either concurrent or future use by the processor 32 or another component of the wireless earpiece 10 and in some embodiments one or more of the memories 34 may be integrated with the processor 32 for improved functionality. Each memory 34 may have activity data , exercise data, nutrition data, fitness programs, environmental data, health monitoring programs, databases, sensor readings related to non-health related data, signals encoding information received from a third-party electronic device, or other types of data or information stored thereon.

An air microphone 44 may be operatively connected to the processor 32 and may be used for providing voice commands to the wireless earpiece 10 or for receiving ambient sounds which may be stored within one or more of the memories 34 by the processor 32. In one embodiment, the air microphone 44 may be outwardly facing on an exterior surface of the wireless earpiece 10 for improved reception. The air microphone 44 may include components such as analog-to-digital converters, amplifiers, attenuators, filters, and/or other components necessary for the air microphone 44 to convert a sound wave into an electrical signal. Voice commands received by the air microphone 44 may be used by one or more programs or applications executed by the processor 32 for controlling one or more functions of the wireless earpiece 10 or for establishing an identity of a user. In addition, ambient sounds received by the air microphone 44 may be used by the processor 32 in lieu of or addition to readings from the sensors 36. Similarly, the bone microphone 45 or conduction microphone may sense vibrations communicated through the bones and other hard tissues of the user's head and body. As a result, the bone microphone 45 may also receive user input (as is subsequently described).

The air microphone 44 or bone microphone 45 may identify activities including high risk actions, such as motorcycle riding, parachuting, snowmobiling, and so forth. The air microphone 44 may also identify low risk activities, such as driving, concerts, biking, swimming, and so forth. For example, the processor may execute one or more programs or applications to compare heart beat sounds received by the air microphone 44 with other activity data, including heart beat data sensed by a sensor 16, for corrective or analytical purposes. If heart beat data sensed by the air microphone 44 does not substantially match with activity data sensed by the heart rate sensor 38, then the processor 32 may delete the data so it is not communicated to the server platform 12 or the mobile application 16. Alternatively, the processor 32 may use a program or application to determine the probability that each data set is faulty. If one data set passes while the other data set fails, the processor may store the data set that passes in a memory 34 and delete the other data set.

In addition, the processor 32 may also associate ambient sounds received by the air microphone 44 with activity data received by a sensor 36 for activity tracking identification purposes. For example, the processor 32 may execute one or more programs or applications to compare a pedaling sound received by the air microphone 44 with a model sound of a person pedaling or riding a bicycle. If the sounds substantially match, then the processor 32 may associate activity data received by a sensor 36 during the time the ambient sounds were received by the air microphone 44 with riding a bicycle. The user may also be prompted to provide data or additional context associated with data, health or otherwise, sensed by the sensor 36. For example, the processor 32 may instruct a speaker 58 to communicate a query to the user to provide information regarding activity that was sensed by one or more sensors 36 over a specific time period. The user may respond to the query via the air microphone 44 by stating “I was jogging around Central Park,” which the processor 32 may associate with the activity sensed by the sensor 36 during the specific time period. Additional context provided by the user, such as the temperature, specifics regarding the activity, geographical features, time of day, or other information which may be pertinent to an insurance provider, may also be associated with the activity by the processor 32. The sensors may also include any number of global positioning, navigation, or location components for identifying the location, motion, orientation, speed, heading, and other information of the user.

The sensors 36 may generate a two or three dimensional scan or topography map of the user's ear and surrounding areas when the wireless earpiece 10 is properly positioned. The mapping may include the internal and/or external portions of the user's ear. The topographical image of the user's ear may be utilized as a stand-alone biometric identifier or may be utilized with other biometric identifiers to identify the user prior to collecting activity data 26 and communicating the data to the server platform 12 or computing device 14. This allows the insurance provider to ensure that the activity data 26 collected corresponds to the holder of the insurance policy or the individual applying for life insurance or health insurance policy. The sensors 36 may also use the air microphone 44 to determine the identity of the user through voice characteristics such as voice frequency, voice altitude, vocabulary, etc. The wireless earpiece 10 may collect a user's heart rate utilizing the heart rate sensor 38 and compare the heart rate to a baseline heart rate to verify identity. In other aspects of the present invention additional or other biometric identifiers such as hand gestures, designated motions, skin conductivity, blood oxygenation, temperature, calories expended per time period, sweat levels, orientation, position, and so forth may be used to verify identity. The sensors 36 may use biometric identifies to verify the identity of the user periodically while a user is wearing the wireless earpiece 10. Allowing insurance providers to verify the user isn't swapping the wireless earpiece 10 with a secondary user to lower the cost of the insurance premium.

The wireless earpiece 10 may include a user interface 60 operatively connected to the processor. The user interface may include a fingerprint scanner that may be utilized to scan a fingerprint (e.g., the index finger) of a user to authenticate a transaction. The user interface 60 may store identifying information for one or more fingers. In one embodiment, the biometric data of the user may be encrypted and stored within a secure portion of the memory 34 to prevent unwanted access or hacking. The wireless earpiece 10 may also tore important biometric data, such as medical information (e.g., medical conditions, allergies, logged biometrics, contacts, etc.) that may be shared in response to an emergency.

A gesture interface 46 may be operatively connected to the processor 32 and may be configured to allow a user to control one or more programs, applications, or functions of the wireless earpiece 10. The gesture interface 46 may include at least one emitter 62 and at least one detector 64 to detect gestures from either the user, a third-party, an instrument, or a combination of the aforementioned and communicate one or more signals representing the gesture to the processor 32. In one embodiment, the gesture interface 46 may include an infrared sensor and a touch/contact sensor. The gestures that may be used with the gesture interface 46 to control the wireless earpiece 10 include, without limitation, touching, tapping, swiping, use of an instrument, or any combination of the aforementioned gestures. Touching gestures used to control the wireless earpiece 10 may be of any duration and may include the touching of areas that are not part of the gesture interface 46. Tapping gestures used to control the wireless earpiece 10 may include any number of taps and need not be brief. Swiping gestures used to control the wireless earpiece 10 may include a single swipe, a swipe that changes direction at least once, a swipe with a time delay, a plurality of swipes, or any combination of the aforementioned. In one embodiment, the gestures, swipes, or taps need not physically contact the gesture interface 46, but may be proximate the gesture interface or made against a portion of the body of the user (e.g., the ear, head, etc.). An instrument used to control the wireless earpiece 10 may be electronic, biochemical or mechanical, and may interface with the gesture interface 34 either physically or electromagnetically.

The speaker 58 may be operatively connected to the processor 32 and may be mounted to or integrated with the earpiece housing 30. The speaker 40 may be positioned in an area on the wireless earpiece 10 conducive for communicating sounds to the tympanic membrane of the user's ear. The speaker 58 may include one or more speakers including tweeters, mid-range, and bass speakers. The speaker 58 may include components such as digital-to-analog converters, amplifiers, attenuators, filters, and/or other components necessary for the speaker 58 to convert an electrical signal into a sound wave. Communications from the speaker 58 may include notifications if the user satisfies activity goals for insurance coverage, a premium adjustment, deductible adjustment, or alerts or warnings if the user fails to satisfy an activity goal, suggestions on how the user may better satisfy an activity goal for a reduced insurance premium, an insurance adjustment or changed qualifications, information regarding activity data used in determining whether to make a premium adjustment (e.g. a heart rate or a blood sugar level), questions associated with an insurance evaluation, notifications whether information was communicated to an insurance provider, notifications regarding whether information was successfully received by an insurance provider, notifications regarding missing information in a user profile, request confirmations, and other information that may be of importance to the user. The components of the speaker 58 may be further configured and/or programmed to generate three-dimensional stereo sound or to generate sounds at specific frequencies.

The wireless earpiece 10 may include light emitting diodes (LEDs) 66 may be operatively connected to the processor 32. The LEDs 66 may be semiconductor-based light sources and may include displays, touch sensors, and/or other interactive interface components. In addition, the LEDs 66 may be configured to provide information concerning the wireless earpiece 10. For example, the processor 32 may communicate a signal encoding information related to the current time, the energy level of the wireless earpiece 10, the status of another operation of the wireless earpiece 10, or another earpiece program or function to the LEDs 66. If the signal concerns the energy level of the earpiece 10, the LEDs 66 may decode the signal as a colored light, wherein a blue light may represent a full battery, a green light may represent a high level of battery life, a yellow light may represent an intermediate level of battery life, a red light may represent a limited amount of battery life, and a blinking red light may represent a critical level of battery life requiring immediate recharging. In addition, the battery life may be represented by the LEDs 66 as a percentage of battery life remaining or may be represented by an energy bar having one or more LEDs, wherein the number of illuminated LEDs represents the amount of battery life remaining in the earpiece. The LEDs 66 may be located in any area on the wireless earpiece 10 suitable for viewing by the user or a third party and may also consist of as few as one diode which may be provided in combination with a light guide. In addition, the LEDs 66 need not have a minimum luminescence.

Energy source 68 may be operatively connected to each of the aforementioned wireless earpiece 10 components and may provide enough power to operate the wireless earpiece 10 for a reasonable duration of time. The energy source 68 may be of any type suitable for powering the wireless earpiece 10. In one embodiment, the energy source 68 is a rechargeable battery, such as a Lithium graphene battery. In another embodiment, the energy source 68 is a solar cell, fuel cell, piezo electric generator, thermal generator, or so forth. Alternative battery-less power sources, such as sensors 36 or inductors configured to receive energy from radio waves or other types of electromagnetic radiation, may be used to power the wireless earpiece 10 in lieu of or in addition to the energy source 68.

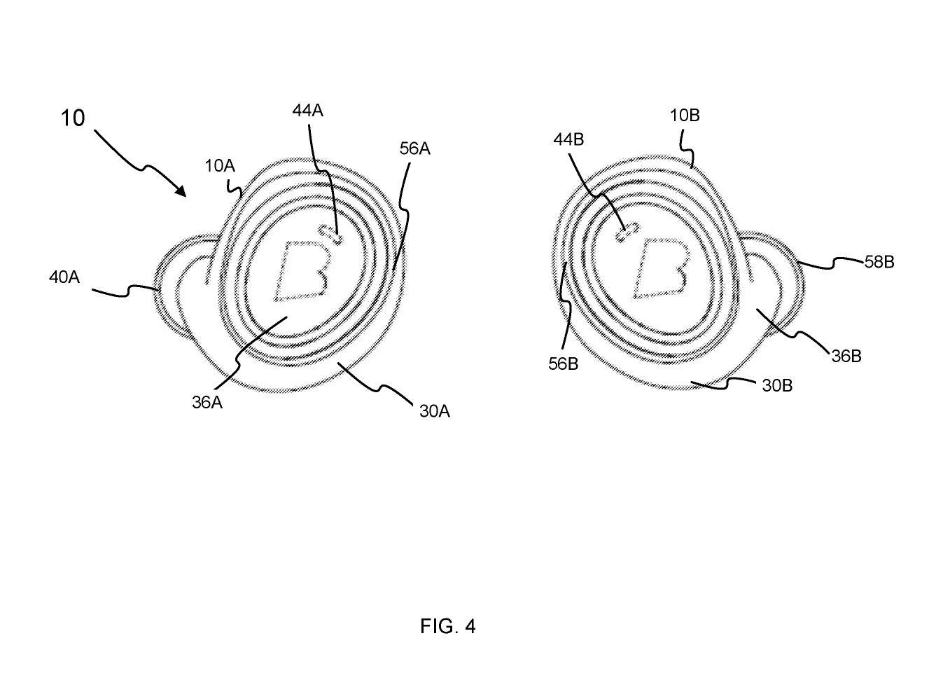

FIG. 4 illustrates another aspect of wireless earpiece 10 which includes a left wireless earpiece 10A and a right wireless earpiece 10B. The left wireless earpiece 10A has a left earpiece housing 30A. The right wireless earpiece 10B has a right earpiece housing 30B. The left earpiece 10A and the right earpiece 10B may be configured to fit on, at, or within a user's external auditory canal and may be configured to substantially minimize or completely eliminate external sound capable of reaching the user's tympanic membranes.

The earpiece housings 30A and 30B may be composed of any material with substantial deformation resistance and may also be configured to be soundproof or waterproof. Sensors 36A and 36B are shown. Sensors 36A and 36B are configured for sensing user actions and activities whether intentional (e.g. running) or unintentional (e.g. a heartbeat or a blood oxygen level) that potentially correspond with activity data that may be of interest to an insurance provider. The sensors 36A, 36B may also measure environmental data. Sensors 36A and 36B may be a physiological sensor (e.g. infrared sensor, optical sensor, a pulse oximeter, heart rate sensor), a motion sensor (e.g. an accelerometer, gyroscope, GPS), or another type of sensor useful for sensing health and environmental data of interest to an insurance provider. The sensors 36A and 36B may be internally facing and externally facing.

Wireless transceivers 56A and 56B are also shown. Each wireless transceiver 56A, 56B is configured to both transmit signals to and receive signals from an server platform, platform, equipment, component, or other electronic device or users. The signals either transmitted by a wireless transceiver or received from an server platform may encode information (e.g. packet communications), a connection frequency, or a waveform in which to connect to and communicate with the other device. Air microphones 44A and 44B are also shown. The air microphones 44A and 44B may be externally positioned to receive one or more sounds or voice commands from the user, one or more sounds from a third party, or one or more ambient sounds from an object worn or carried by the user or the outside environment, whether natural or artificial.

A speaker 58A is shown on the left wireless earpiece 10A and a speaker 58B is shown on the right wireless earpiece 10B. Speakers 58A and 58B may be positioned proximate to the user's tympanic membranes for communicating notifications, alerts, warning, suggestions, or other important information to the user. Speakers 58A and 58B may also be configured to short out if the any sounds exceed a certain decibel threshold, which may be preset or programmed by the user or a third party.

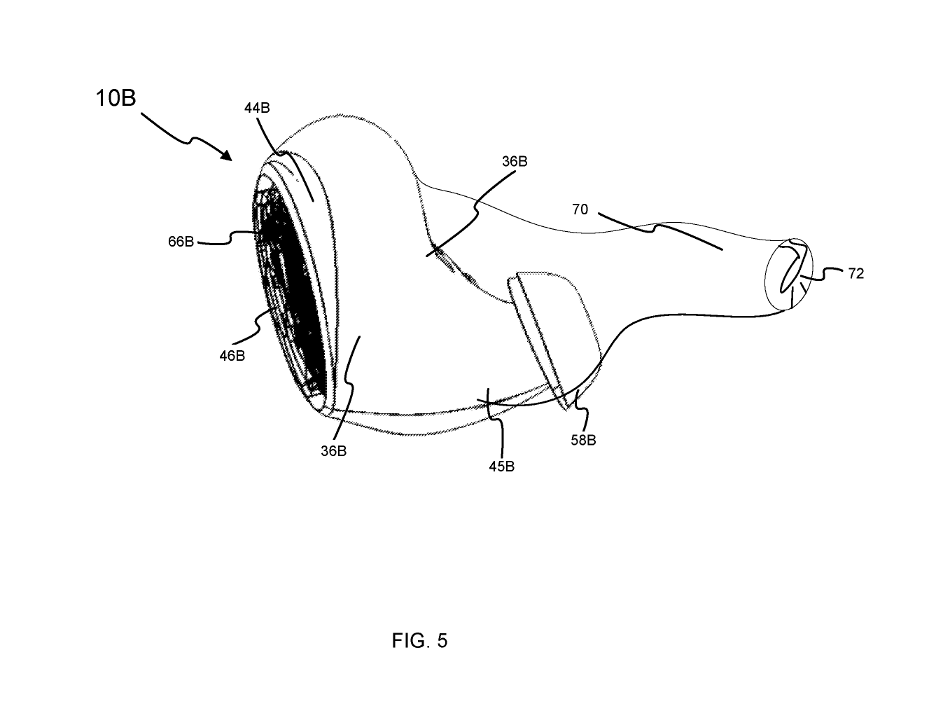

FIG. 5 illustrates a side view of the right wireless earpiece 10B and its relationship to the user's ear. The right wireless earpiece 10B may be configured to both minimize the amount of external sound reaching the user's external auditory canal 70 and to facilitate the transmission of sound from the speaker 40B to a user's tympanic membrane 72. The speaker 58B may utilize active or passive noise cancellation. For example, the speaker 58B may communicate a noise cancellation signal generated by the right wireless earpiece 10B in response to exterior sound detected by the air microphone 44B.

The right wireless earpiece 10B may also be configured to be of any size necessary to comfortably fit within the user's external auditory canal 70. A gesture interface 46B may be positioned on the outside of the right wireless earpiece 10B and may provide for touch or gestural control by the user or a third party such as by tapping or swiping across (or in front of) the gesture interface 46B, tapping or swiping across another portion of the right earpiece 10B, providing a gesture not involving the touching of the gesture interface 46B or another part of the right earpiece 10B, or through the use of an instrument configured to interact with the gesture interface 46B. The user may use the gesture interface 46B for responding to queries relating to an alert or notification regarding a condition for obtaining a premium adjustment communicated by the speaker 58B or for responding to queries relating to an insurance evaluation. LEDs 66B may also be present, which may be integrated with the gesture interface 46B. A bone conduction microphone 45B may be positioned near the temporal bone of the user's skull in order to receive sounds and vibrations directly from the bone. The sounds received by the bone conduction microphone 45B may include heartbeats, heart palpitations, heart arrhythmias, sounds indicative of a physical activity such as walking, running, jogging, or weightlifting, or other related sounds. The bone conduction microphone 45B may also sense sounds before the sounds reach either the air microphone 44B in order to differentiate between sounds from the user and the ambient.

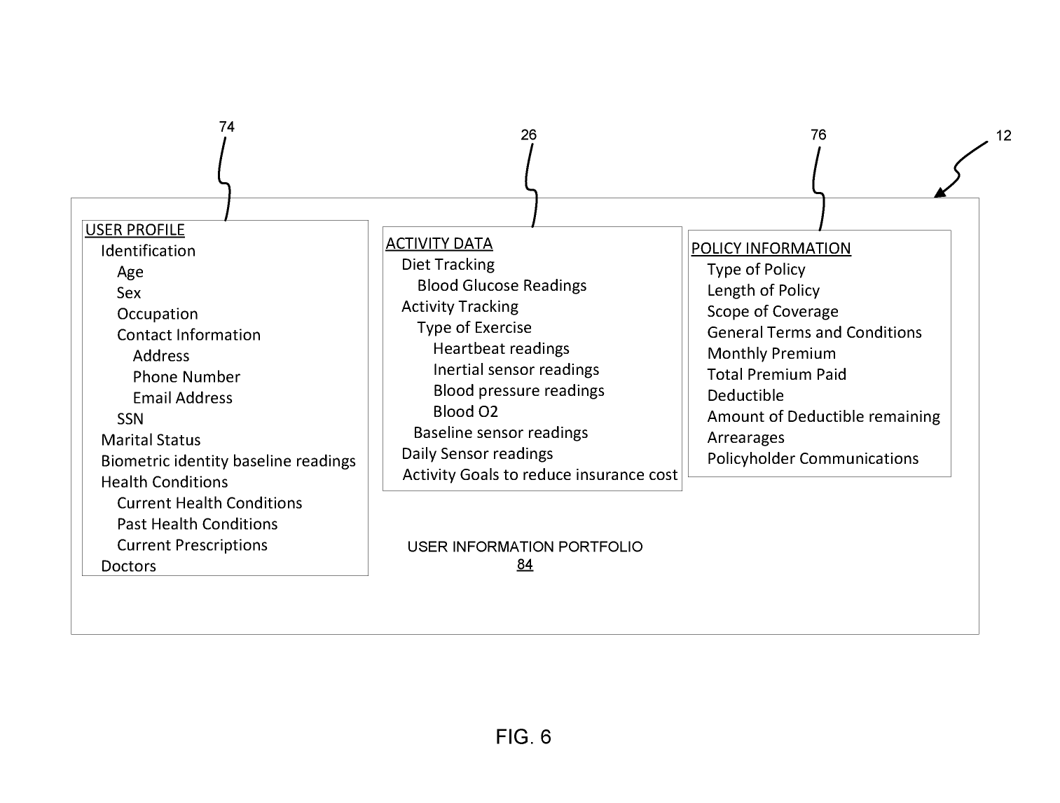

FIG. 6 illustrates one example of a user information portfolio 84 contained within the mobile application 16 or on the server platform 12 for use in determining the insurance premium for the user, reducing the cost of the potential insurance premium or adjusting an existing insurance premium. The information may be utilized for any number of different types of insurance including health insurance, life insurance, driving insurance, or so forth. The mobile application or server may contain a user profile 74. The user profile 74 may include information relating to the identity of the user including the age, sex, and occupation, contact information (address, phone number, email address and any other contact information), marital status, Social Security Number. The user profile 74 may further include biometric identity baseline readings for identity verification, health conditions of the user including current and past health conditions and current prescriptions, and any doctors.

The user information portfolio 84 stored on the server platform 12 or on the mobile application 16 may include activity data 26 communicated to the server platform 12 or the mobile application 16 by the wireless earpiece 10 and other wearable activity tracking devices. The activity data 26 may include diet tracking including blood glucose readings, activity tracking. The activity tracking may include the type of exercise performed, the heart rate readings during the exercise, the inertial sensor readings during the exercise, the blood pressure readings during the exercise and the blood O2 readings during the exercise. The activity data 26 may also include baseline sensor readings, daily sensor readings, and activity goals to reduce insurance cost, or notifications received.

The user information portfolio 84 may contain policy information 76. The policy information 76 may include the current insurance information (e.g., premiums, payments, balances, limits in coverage, legal stipulations, deductibles, thresholds, maximums, minimums, etc.) the user is responsible for (including any arrearages), the amount of the premium that the user has paid, the term of a policy, the type of insurance policy, any terms or conditions that can void the insurance policy, the scope of the coverage, insurance evaluations.

Furthermore, the user information portfolio 84 may include specific information relevant for determining whether the user is eligible for insurance or insurance adjustments. Such specific information may include specific health thresholds related to the user or a health condition of the user, parameters to be used for determining if certain conditions and/or thresholds are being satisfied, warnings for failing to meet certain thresholds (wherein the thresholds may be determined by either the user, the insurance provider, or a third party such as a medical professional), suggestions related to health improvement, specific premium adjustments if certain conditions, whether good or bad, are satisfied, medical conditions related to the user, prescriptions being taken by the user, past prescriptions taken by the user, medical procedures which have been performed on the user, risks factors associated with the user, medical professionals who have medically reviewed the user, requests by the user or the insurance provider, and other health related information which may be pertinent to an insurance provider. The user profile may also include conditions or thresholds related to the user's prescriptions or medical procedures, conditions or thresholds related to the user's diet, conditions or thresholds related to the user's exercise habits, or other conditions or thresholds that may be useful in ascertaining whether a user is entitled to a premium adjustment. The user profile may also include other types of information which may be pertinent to an insurance provider in making a premium adjustment.

User information may be established for any number of users that may utilize the wireless earpieces. In other embodiments, the wireless earpieces may be utilized by insurance or medical professionals for a temporary evaluation of the user. For example, for life insurance and health insurance quotes to be generated a health insurance company may require that the wireless earpieces be worn for specified time periods over an interval (e.g., a week). A health and status of evaluation of the user may be generated based on the measured and analyzed information.

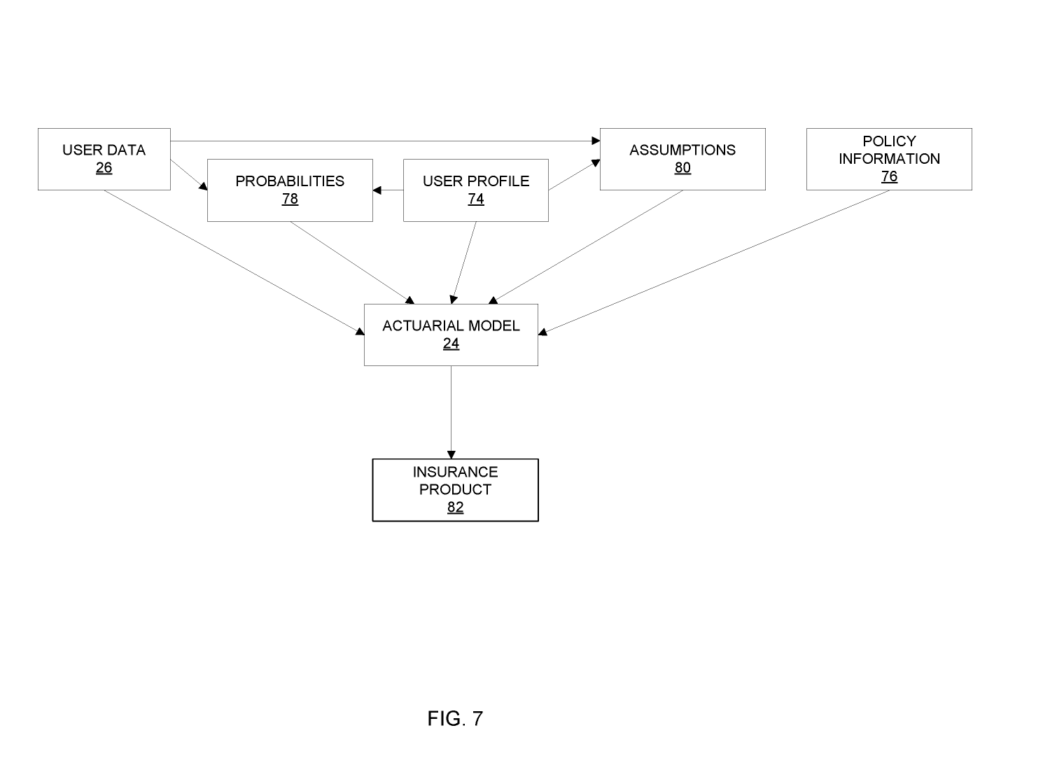

FIG. 7 illustrates another aspect of the server platform 12. The server platform 12 may store activity data 26, probabilities 78, user profile 74, assumptions 78, and policy information 76. This information may be inputted in to an actuarial model 24 to provide an insurance product 82 for the user. The actuarial model 24 may focus predictive modeling by calculating life expectancy of the user, producing life tables and calculating compound interest to produce a life insurance policy for the user. The actuarial model may use user information or data to calculate the level of risk The actuarial model may also focus on analyzing disability rates, morbidity rates, mortality rates, fertility rates, risk of developing diseases, injury risk, and other contingencies such as consumer choice and the geographical distribution of the utilization of medical services and procedures, and the utilization of drugs and therapies when calculating or adjusting a user's health insurance premium.

The actuarial model may make or input certain assumptions 80 regarding a user. The assumptions 80 may use information from the user profile 74 or the activity data 26. The assumptions may include mortality, persistency, morbidity, expenses, earned income, claims, lapses in coverage.

The actuarial model may make or input certain probabilities 78 regarding the user when determining a premium cost. The probabilities 78 may use information from the user profile 74 or the activity data 26. The probabilities 78 may include life expectancy, incurring certain illness or disease, risk of other injuries, becoming disabled, mortality, active or sedentary lifestyle.

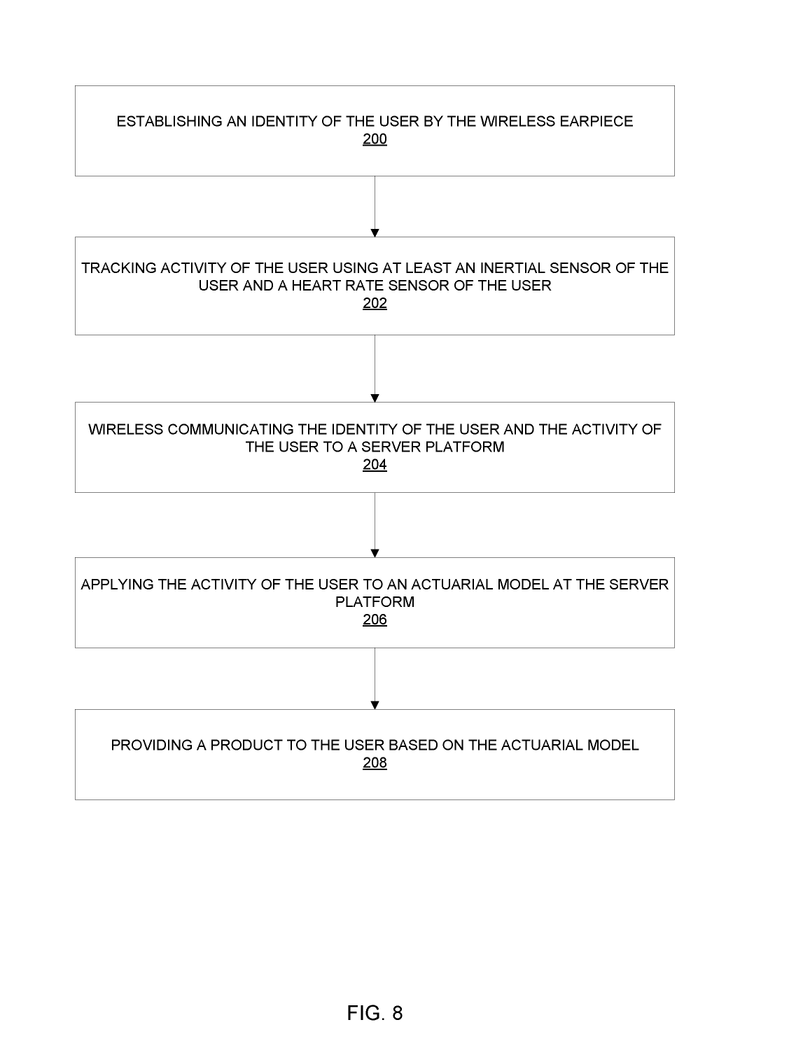

FIG. 8 illustrates one method of sharing information utilizing the wireless earpiece with an insurance provider. First, the wireless earpiece 10 establishes the identity of the user (step 200). The wireless earpiece may use biometric data received at the processor 32 by the sensors 16. The wireless earpiece may also use passcodes, passwords, gestures or any other verification method to establish the identity of the user.

Next the wireless earpiece tracks the activity of the user utilizing sensors 16 of the wireless earpiece (step 202). In step 202, a sensor 36 associated with a wireless earpiece receives one or more pieces of data associated with a user. The sensor readings may be performed continuously or discretely depending on the settings of the relevant program or the requirements of the insurance provider. In addition, more than one type of sensor 36 may be used, and each sensor may sense a different parameter associated with the user. For example, one sensor may be a pulse oximeter configured to measure blood oxygen levels and another sensor may be an internal bone conduction microphone 45 configured to measure the user's heart rate. Additional sensors and sensor types may also be employed to measure other relevant health and biometric parameters. The sensors 36 of the wireless earpieces 10 may also measure environmental information associated with the user including, but not limited to, noise levels, air quality/composition, proximity to users/objects, speed, location, heading, radiation, gas levels, sunlight, UV exposure, water exposure, and so forth. The wireless earpiece 10 may use the heart rate sensor 38 and the inertial sensor 40 to track the heart rate and movement of the user.

A user's activity may also be tracked daily to determine how active or sedentary a user's lifestyle is. The wireless earpiece may incorporate one or more sensor readings while tracking activity. The wireless earpiece may incorporate sensor readings from another wireless earpiece 10 or an additional activity tracking device.

The insurance provide may offer certain discounts or other financial benefits if a user meets certain activity goals tracked by the wireless earpiece 10. The goals may be communicated to the wireless earpiece 10 from the server platform 12 directly or through the mobile application 16 on the computing device 14. The speaker 58 may communicate the activity goals to the user. The user may also be notified via the speaker 58 of progress towards completing an activity goal, failure to complete an activity goal, or completion of an activity goal. The insurance provider may lower the cost of the insurance product 82 if the user exercises 30 minutes a day, 5 days a week. The insurance provide may lower the cost or deduct a percentage or dollar amount from the cost of the insurance product of the user engages in a more active lifestyle, which may include walking 10,000 steps a day, elevating heart rate at least once a day for a set period of time, etc. The insurance provider may also view the activity tracking date at the end of each month or year to determine whether the cost of the insurance product should be adjusted, reduce or increased.

Next, the wireless earpiece 10 wirelessly communicates the identity of the user and the activity of the user to a server platform 12 (step 204). The wireless earpiece 10 may communicate activity data 26 using the transceiver 42 to the server platform 12. The wireless earpiece may also communicate the activity data 26 to a computing device 14. The computing device may have a mobile application 16 provided by the insurance company to store activity data 26, user profile 74 and policy information 76. The computing device may then wireless communicate the user information to the network of the server platform 12.

Next the server platform 12 may apply the activity of the user to an actuarial model 24 (step 206). The actuarial model may use user information from the user profile 74 or the activity data 26 to calculate the level of risk. The actuarial model 24 may also focus on analyzing disability rates, morbidity rates, mortality rates, fertility rates, risk of developing diseases, injury risk, and other contingencies such as consumer choice and the geographical distribution of the utilization of medical services and procedures, and the utilization of drugs and therapies when calculating or adjusting a user's health insurance premium.

Next the user is provided with an insurance product (step 208). The insurance product may be based on the user activity. The insurance product may include a life insurance product, a health insurance product or another type of insurance product.

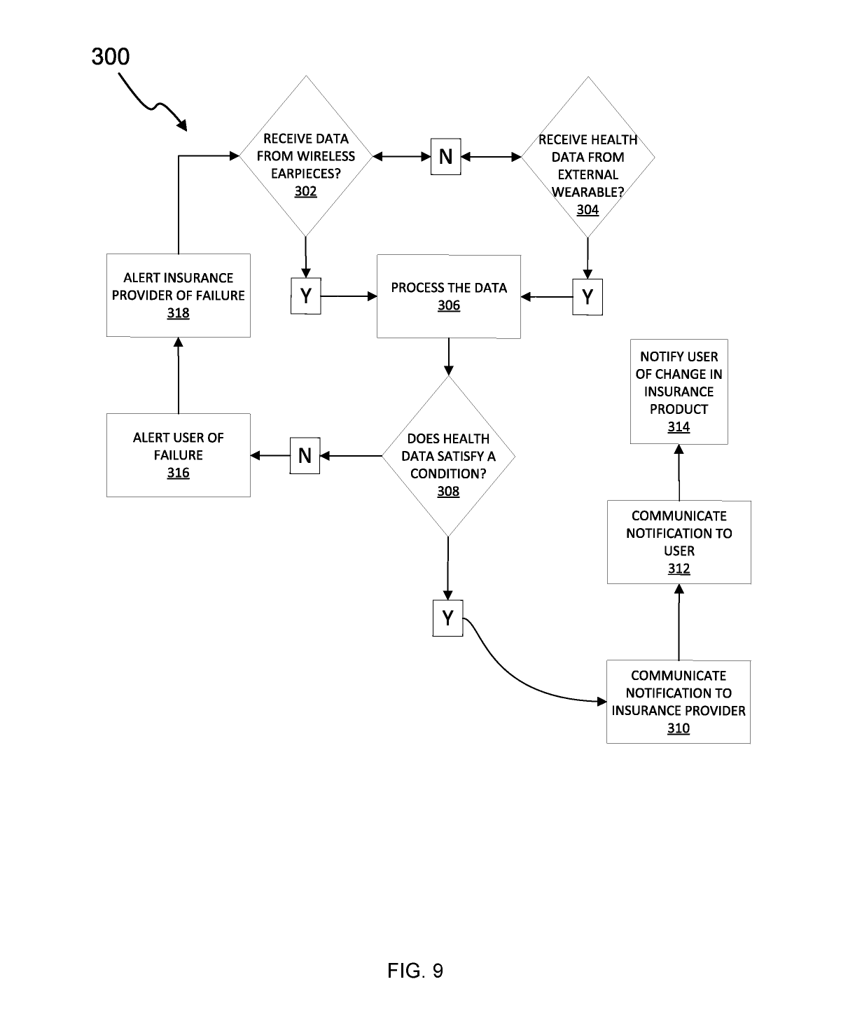

FIG. 9 illustrates one embodiment of a method 300 sharing data with an insurance provider. In step 302, a sensor 36 associated with a wireless earpiece 10 receives one or more pieces of data associated with a user. The sensor readings may be performed continuously or discretely depending on the settings of the relevant program or the requirements of the insurance provider. In addition, more than one type of sensor 36 may be used, and each sensor may sense different biometric data associated with the user. For example, one sensor may be a pulse oximeter configured to measure blood oxygen levels and another sensor may be an internal bone conduction microphone configured to measure the user's heart rate. Additional sensors and sensor types may also be employed to measure other relevant health and biometric parameters. The sensors 36 of the wireless earpieces 10 may also measure environmental information associated with the user including, but not limited to, noise levels, air quality/composition, proximity to users/objects, speed, location, heading, radiation, gas levels, sunlight, UV exposure, water exposure, and so forth.

In step 304, information from additional wearable devices worn by the user may be received by the wireless earpiece. For example, if the user is wearing a smart watch, the smart watch may measure the user's heart rate and transmit a signal encoding the heart rate data to the wireless earpiece. The heart rate data may be communicated contemporaneously with the reception of the data or at a later time. The information received from the additional wearable devices may be incorporated with activity data received by the wireless earpiece 10 if the signal encoding the heart data includes temporal information which can be used by a processor 32 of the wireless earpiece 10 to associate the information with the activity data . Data from any number of smart phones, bands, implantable devices, smart stickers/adhesives, vehicle systems, environmental monitors, or so forth may also be utilized.

In step 306, the activity data 26 sensed or received by the sensor 36 of the wireless earpiece and, if applicable, any additional activity related data or information received from another wearable device is processed by the processor 32 associated with the wireless earpiece 10. The processing may be performed in accordance with one or more parameters set or desired by an insurance provider. For example, if the insurance provider provides a premium discount or a new insurance product if the user performs an acceptable form of physical activity for a certain number of hours each month, the processor 32 may determine the amount of time the user spent performing a physical activity by comparing heartbeat measurements against the user's baseline resting heart rate. The heartbeat measurements may come from the wireless earpiece 10, wearable device, or other user-associated electronics.

If the processor 32 determines that the user had an elevated heart rate over one or more time periods that may be indicative of physical activity, then the processor may search a memory 34 operatively connected to the processor or within a component of the processor itself to determine whether sounds received at a sensor 36 (such as a microphone) during each time period the user had an elevated heart rate correspond to a physical activity. Sounds received during each time period may be compared to a sample jogging sound, a sample running sound, or another sound related to physical activity to determine the likelihood of whether the sounds are indicative of physical activity performance. The likelihood and standard error required to satisfy whether the user performed an acceptable physical activity may be preset by the program, application, or algorithm used to determine whether sounds received at a sensor substantially match a model sound or the likelihood may be set by the insurance provider. As another example, if the insurance provider provides a premium discount if the user consumes under a certain amount of carbohydrates (e.g. if the user is diabetic), the processor may use sensor readings obtained from a blood glucose sensor to determine whether the user has satisfied the condition. The sensor readings may originate from the wireless earpiece 10 or another wearable device of the user.

In step 308, the processor 32 of the wireless earpiece 10 determines whether the data sensed by the wireless earpiece or another wearable device satisfies one or more activity goals required by the insurance provider and communicated to the wireless earpiece 10 from the server platform 12 for performing an insurance related option (e.g., premium/deductible adjustment, quote estimate, insurance offer, maximum increase, etc.). If the processor 32 determines that at least activity goal has been satisfied, then in step 310, a signal encoding that the user satisfied a condition is communicated to an insurance provider. The signal may also encode that the user satisfied one or more additional conditions as well. For example, a message encoded in one more data packets may be communicated. If the insurance provider does not receive the signal or the insurance provider communicates that the signal was not received, then the signal may be retransmitted as many times as necessary. Contemporaneously with the communication of the signal or after the communication, in step 312, a notification is communicated via a speaker associated with the wireless earpiece to the user. The communication may also be communicated before the communication of the signal encoding the satisfaction of one or more conditions to the insurance provider. In addition, one or more suggestions or warnings may be communicated to the user as well. For example, if the user satisfied one condition, but failed to satisfy another condition (e.g. the user exercised but failed to maintain an acceptable diet), a communication providing examples of foods and beverages that can help satisfy the failed condition and foods and beverages that can exacerbate the failed condition may be provided.

If the insurance provider receives the signal in step 310, then in step 314, a notification from the insurance provider may be communicated to the wireless earpiece that the user's insurance is to be adjusted. The notification may also be communicated to the computing device 14 of the user. The user may also view the notification using the mobile application 16. The notification may also include information regarding insurance qualifications, a premium/deductible adjustment for future months, new insurance products available, new guidelines or thresholds required to satisfy a condition for reducing the cost of the insurance product, news regarding the user's health, adjusted costs/expenses, risks/exposure, or other information relevant to insurance.

If the processor determines that the user failed to satisfy any conditions, then in step 316, an alert is communicated via the speaker of the wireless earpiece informing the user of the failure. The information provided to the user may include parameters or thresholds required to satisfy one or more conditions, suggestions for improvement, potential consequences if the user fails to improve, health information related to the user, health comparisons to users with substantially similar characteristics, percentiles related to the health comparisons, and other information that can be of use to the user for improvement. Contemporaneously with the communication of the alert, in step 318, a notification is communicated to the insurance provider informing the insurance provider that the user failed to meet any condition required for an insurance change. The communication may also be sent before or after the communication of the alert.

The features, steps, and components of the illustrative embodiments may be combined in any number of ways and are not limited specifically to those described. In particular, the illustrative embodiments contemplate numerous variations in the smart devices and communications described. The foregoing description has been presented for purposes of illustration and description. It is not intended to be an exhaustive list or limit any of the disclosure to the precise forms disclosed. It is contemplated that other alternatives or exemplary aspects are considered included in the disclosure. The description is merely examples of embodiments, processes or methods of the invention. It is understood that any other modifications, substitutions, and/or additions may be made, which are within the intended spirit and scope of the disclosure.

The previous detailed description is of a small number of embodiments for sharing activity data utilizing a wireless earpiece with an insurance provider and is not intended to be limiting in scope. The following claims set forth a number of the embodiments of the invention disclosed with greater particularity.

What is claimed is:

1. A method for sharing user information with an insurer, the method comprising:
establishing an identity of the user by a wireless earpiece of the user;
tracking the activity of the user using at least an inertial sensor of the user and a heart rate sensor of the user:
wirelessly communicating the identity of the user and the activity of the user to a server platform;
applying the activity of the user to an actuarial model at the server platform;
wherein an insurance product is provided to the user based on the actuarial model.

2. The method of claim 1 further comprising:
providing feedback to the user through the wireless earpiece indicative of progress towards an activity goal based on the activity of the user wherein the activity goal is associated with an insurance adjustment.

3. The method of claim 1 further comprising:
communicating from a transceiver of the wireless earpiece the activity of the user to a mobile application on a computing device of the user.

4. The method of claim 3 further comprising:
communicating from the mobile application on the computing device of the user over a wireless communications network to the server platform.

5. The method of claim 1 wherein the activity of the user is tracked while the user is performing exercise.

6. The method of claim 1 wherein the identity of the user is established using biometric data received by at least one sensor of the wireless earpiece.

7. The method of claim 6 wherein the biometric data includes at least a voice characteristic.

8. The method of claim 6 wherein the biometric data includes data from the heart rate sensor.

9. The method of claim 6 wherein the biometric data include a topographical map of a portion of an ear of the user.

10. The method of claim 1 wherein the insurance product is a life insurance product.

11. The method of claim 1 wherein the insurance product is a health insurance product.

12. The method of claim 1 further comprising:
communicating activity goals from the server platform through a wireless network to the computing device of the user.

13. The method of claim 12 further comprising:
associating activity data communicated from the wireless earpiece to the computing device with the activity goals stored on the mobile application.

14. The method of claim 1 wherein a processor of the wireless earpiece analyzes the activity data.

15. The method of claim 14 wherein the processor of the wireless earpiece determines whether the activity data of the user satisfies an activity goal communicated from the server platform.

16. The method of claim 1 wherein a processor of the wireless earpiece notifies the user through a speaker of the wireless earpiece that the user completed an activity goal communicated to the wireless earpiece from the server platform.

17. The method of claim 1 wherein a speaker of the wireless earpiece notifies the user of the insurance product.

18. The method of claim 17 wherein the speaker of the wireless earpiece notifies the user changes to the insurance product.

19. The method of claim 1 wherein the wireless earpiece transmits a signal to the server platform to establish a connection to server platform.
